(19) United States
(12) Patent Application Publication
(10) Pub. No.: US 2017/0369928 A1
(43) Pub. Date: Dec. 28, 2017

(54) METHOD FOR EVALUATING DNA DAMAGE FROM ANALYTE

(71) Applicant: SOGANG UNIVERSITY RESEARCH FOUNDATION, Seoul (KR)

(72) Inventor: Kyu Bong JO, Gyeonggi-do (KR)

(73) Assignee: SOGANG UNIVERSITY RESEARCH FOUNDATION

(21) Appl. No.: 15/392,986

(22) Filed: Dec. 28, 2016

(30) Foreign Application Priority Data
Jun. 27, 2016 (KR) 10-2016-0080274

(51) Int. Cl. C12Q 1/68; C12N 11/04

(52) U.S. Cl. CPC C12Q 1/6806; C12N 11/04; C12N 2533/76

(57) ABSTRACT

The present disclosure provides a method for evaluating DNA damage by an analyte and a method for screening a DNA damage inhibitor. According to the present invention, the present invention can quantitatively evaluate the extent of DNA damage by an analyte through visualization.

### CROSS-REFERENCE TO RELATED APPLICATIONS

This application claims the benefit and priority of Korean Patent Application No. 10-2016-0080274, filed Jun. 27, 2016. The entire disclosure of the above application is incorporated herein by reference.

### FIELD

The present disclosure relates to a method for evaluating DNA damage from analyte.

### BACKGROUND

The prevention and repair of DNA damage is imperative for the survival of living organisms. DNA damage is the leading cause of many health risks and is directly correlated with different health conditions. Moreover, it is the primary cause of accelerated aging, resulting in a variety of diseases including cancer. In addition to ultraviolet and environmental pollution, food digestion and naturally occurring endogenous metabolites also cause DNA damage. For example, alcoholic beverages produce a considerable amount of reactive oxygen species (ROS) that damage DNA. The World Health Organization (WHO) global burden of disease (GBD) project reported that chronic alcohol consumption accounts for ˜1.8 million deaths per year. Cancer is one of long-term effects of alcohol drinking. Scientists reported that chronic alcohol consumption might be related to malignant tumours in the liver, breast, and gastrointestinal tract. However, there have been contradictory reports. For instance, red wine has both cancerous and anti-cancerous properties. These controversial results may be attributed to analytical methods not being straightforward enough to clearly evaluate the effects of a certain food. Furthermore, existing methods for evaluating the health risks of a food are based on other principles and are not considerate for DNA damage. Therefore, a novel sensitive assay is needed to properly evaluate the beneficial or harmful effects of a particular food.

Although numerous methods have been developed for analyzing DNA damage, single molecule DNA visualization has recently emerged as the most sensitive means to detect DNA damage, because the number of damaged lesions can be directly visualized and quantified. DNA analysis methods, in general, are based on DNA amplification. However, damaged DNA cannot be amplified, leading to a severe limitation in these methods. Thus far, DNA damage has been analyzed by measuring the smearing pattern in the gel, including comet assay that uses single cells and the DNA laddering assay that uses apoptotic DNA fragmentation. However, smeared pattern does not provide detailed information. In addition, DNA damage occurs very rarely as a chronic process, making it difficult for electrophoresis-based assays to detect these rare events. Therefore, single molecule detection is the most optimal method for DNA damage analysis owing to its high sensitivity. Furthermore, single molecule DNA damage detection has great potential for development as an analytical biosensor to quantitatively evaluate food-induced DNA damage.

Throughout the entire specification, many papers and patent documents are referenced and their citations are represented. The disclosure of the cited papers and patent documents are entirely incorporated by reference into the present specification and the level of the technical field within which the present invention falls, and the details of the present invention are explained more clearly.

### SUMMARY

The present inventors endeavored to construct a DNA damage analysis system using microorganisms. As a result, the present inventors established the single-molecule visualization by preparing an Escherichia coli (the representative bacterium)-embedded gel, bringing the gel into contact with an analyte to induce DNA damage, and fluorescently labeling the damaged DNA, and thus completed the present invention.

Therefore, an aspect of the present invention is to provide a method for evaluating DNA damage by an analyte.

Other purposes and advantages of the present invention will become more obvious with the following detailed description of the invention, claims, and drawings.

In accordance with an aspect of the present invention, there is provided a method for evaluating DNA damage by an analyte, the method including:
(a) culturing cells to obtain a cell suspension;
(b) gelating the cell suspension to prepare a cell-embedded gel;
(c) bringing the cell-embedded gel into contact with an analyte;
(d) lysing the cell-embedded gel;
(e) performing DNA glycosylase treatment on the product of step (d);
(f) labeling the product of step (e) through nick translation;
(g) isolating genomic DNA from the product of step (f); and
(h) analyzing the genomic DNA.

The present inventors endeavored to construct a DNA damage analysis system using microorganisms. As a result, the present inventors established the single-molecule visualization by preparing an Escherichia coli (the representative bacterium)-embedded gel, bringing the gel into contact with an analyte to induce DNA damage, and fluorescently labeling the damaged DNA.

The method for evaluating DNA damage by an analyte of the present invention will be described by steps.

Step (a): Obtaining of Cell Suspension

First, cells are cultured to obtain a cell suspension.

As the cells, any cells that are known in the art may be used.

According to an embodiment, the cells are selected from the group consisting of microorganisms, animal cells, and plant cells.

The microorganism refers to a living organism composed of single cells and a minimum living unit of a living organism. The microorganism includes alga, bacteria, protozoa, molds, yeasts, and viruses, but is not limited thereto.

The animal cell includes all cells immediately after the isolation from living animal living bodies or tissues, initially cultured cells, and established cell lines, and, for example, includes mammals including humans, birds, fish, and insects, but are not limited thereto.

The plant cell includes all cells immediately after the isolation from organs of plants, initially cultured cells, and established cell lines.

According to another embodiment of the present invention, the cells are derived from microorganisms.

According to a particular embodiment of the present invention, the cells are derived from bacteria.

As used herein, the term “cell suspension” may be any culture broth containing living cells.

According to an embodiment of the present invention, the cell suspension is a suspension obtained by harvesting cells from a culture broth containing a culture medium and suspending the cells in a sterile saline solution (0.85% NaCl solution).

According to another embodiment of the present invention, the cells are log-phase or exponential phase cells showing the highest rate of growth.

As proved in the following examples, the cell suspension has an OD₆₀₀ value of 0.5.

Step (b): Preparation of Cell-Embedded Gel

The cell suspension is gelated to prepare a cell-embedded gel.

According to an embodiment of the present invention, the gelation is performed by adding, to the cell suspension, at least one polymer selected from the group consisting of agarose, agar, carrageenan, gellan gum, gelatin, pectin, alginate, fibrin, polyacrylate, polyethylene glycol, chitosan, dextran, collagen, and hyaluronic acid.

According to another embodiment of the present invention, the gelation is performed by adding agarose to the cell suspension.

As proved in the following example, the gelation is induced by adding, to the cell suspension, a 2% low-gelling temperature (LGT) agarose in drops. The term “agarose plug” in the following examples has the same meaning as the “cell-embedded gel”.

The cell-embedded gel has air holes through which enzymes and small-sized molecules can pass freely.

According to an embodiment of the present invention, the cell-embedded gel has air holes with a size of 10-1000 nm.

According to another embodiment of the present invention, the cell-embedded gel has air holes of 50-1000, 50-700, 50-400, or 50-200 nm.

Step (c): Contact With Analyte

The cell-embedded gel is brought into contact with an analyte.

As used herein, the term “analyte” refers to an unknown material that is used to test whether it influences DNA damage. The analyte includes not only biological materials, including compounds, proteins, nucleotides, antisense nucleotides, siRNA, but also includes artificial materials, including medicines, food, cosmetic products, and agricultural chemicals, but is not limited thereto. That is, in the method for evaluating DNA damage by an analyte of the present invention, any material for evaluating the presence or absence of DNA damage by an analyte and the extent thereof may be used.

As proved in the following examples, the cell-embedded gel is immersed in an analyte (or a composition containing an analyte) to induce DNA damage by the analyte.

Step (d): Lysis of Cells

The cells in the cell-embedded gel are lysed.

According to an embodiment of the present invention, the lysis is performed by the treatment with a lysing agent.

As used herein, the term “lysing agent” refers to a material that disrupts the membrane of cells (e.g., bacteria) to lyse the cells. The lysing agent is at least one selected from the group consisting of an alkali, a surfactant, an organic solvent, and an enzyme (e.g., protease K).

As proved in the following examples, the cell-embedded gel is treated with protease K to lyse the cells.

Step (e): Treatment With DNA Glycosylase

The product of step (d) is treated with DNA glycosylase.

As used herein, the term “DNA glycosylase” is an enzyme associated with base excision repair, and refers to an enzyme group belonging to EC 3.2.2. that removes or substitutes damaged bases in DNA.

According to an embodiment of the present invention, the DNA glycosylase is at least one selected from the group consisting of formamidopyrimidine [fapy]-DNA glycosylase (Fpg), endonuclease IV (Nfo), endonuclease VIII (Nei), 3-methyladenine DNA glycosylase II (AlkA), uracil-DNA glycosylase (UDG), endonuclease III (Nth), adenine DNA glycosylase (MutY), 3-methylpurine DNA glucosylase (AlkC), and akylpurine glycosylase D (AlkD).

According to another embodiment of the present invention, the DNA glycosylase is at least one selected from the group consisting of Fpg, Nfo, and Nei.

According to a particular embodiment of the present invention, the DNA glycosylase is a NDA glycosylase mix of Fpg, Nfo, and Nei.

Fpg acts \textcircled{1} to release damaged purine and generate AP site (apurinic site); and \textcircled{2} to cut the 3′ and 5′ terminals of the AP site and remove the AP site, thereby generating a 1-base gap.

Nfo is 5′ AP endonuclease, and acts to cut the 5′-phosphodiester bond of the base damage site of DNA.

Nei acts to cut the oxidized pyrimidine.

Step (f): Labeling

The product of step (e) is labeled through nick translation.

With respect to the labeling, the single-molecule DNA is labeled by using nick translation, so that the presence or absence of a particular nucleotide sequence can be visualized in the single-molecule level. Through the nick translation, gaps or nicks in the DNA generated in step (e) are filled using DNA polymerase, and genomic DNA may be fluorescently labeled using signal material-labeled dNTPs.

According to an embodiment of the present invention, the labeling is performed by DNA polymerase and signal material-labeled dNTP mix (dATP, dCTP, dGTP, dTTP, and dUTP).

As used herein, the term “DNA polymerase” refers to an enzyme that synthesizes DNA molecules from deoxyribonucleotides as constituent elements of DNA. The DNA polymerase catalysts the following chemical reaction:

Deoxyribonucleotide triphosphate+DNAn⇄diphosphate+DNAₙ₊₁

According to an embodiment of the present invention, the DNA polymerase is endonuclease free DNA polymerase I.

DNA polymerase I mediates nick translation through 5′→3′ exonuclease activity during the DNA damage procedure.

The dNTPs, which are constituent elements of DNA, include deoxyadenosine triphosphates (dATP), deoxyguanosine triphosphates (dGTP), deoxycytidine triphosphates (dCTP), deoxythymidine triphosphates (dTTP), and deoxyuridine triphosphates (dUTP).

The dNTPs are labeled with a suitable signal material. Specific examples of the signal material are as follows: Specific examples of the signal material include fluorophores (e.g., fluorescein, phycoerythrin, rhodamine, lissamine, and Cy3 and Cy5 (Pharmacia)), chromophores, chemiluminophores, magnetic particles, radioisotopes (P32 and S35), mass labels, electron-dense particles, enzymes (alkaline phosphatase or horseradish peroxidase), cofactors, substrates of enzymes, heavy metals (e.g., gold), antibodies, streptavidin, biotin, digoxigenin, and haptens having specific binding partners such as a chelating group, but are not limited thereto. The labels provide signals that can be detected by fluorescence, radioactivity, chromophore measurement, weight measurement, X-ray diffraction or absorption, magnetism, enzymatic activity, mass analysis, binding affinity, hybridization high frequency, and nanocrystals.

As proved in the following examples, the gaps and/or nicks in DNA are labeled by a constant-temperature reaction at 37° C. for 1 hour.

Step (q): Extraction of Genomic DNA

The cell-embeded gel is lysed from the product in step (e), and the resultant product is analyzed.

The cell-embedded gel is lysed to isolate cell genomic DNA. The isolation of cell genomic DNA may be performed by various methods that are known in the art.

Step (h): Analysis of Genomic DNA

The genomic DNA is analyzed.

According to an embodiment of the present invention, the analysis of the genomic DNA is performed using a microfluidic device.

As used herein, the term “microfluidic device” refers to a device that implements a series of techniques of controlling the flow of a trace (nanoliter or picoliter) of liquid or gas in a severely miniaturized device.

According to an embodiment of the present invention, the microfluidic device has a channel, into which a fluid is introduced, and a positively charged substrate directly connected with the channel.

The substrate is selected from the group consisting of glass, plastic, and silicone substrates.

As proved in the following examples, the genomic DNA is injected into the microfluidic channel, and elongated and deposited on a surface having positive charges. Then, the genomic DNA was observed using a fluorescent microscope.

According to the method, the extent of DNA damage by an analyte can be evaluated. This method allows a quantitative evaluation by visualizing the extent of DNA damage.

In accordance with another aspect of the present invention, there is provided a method for screening a DNA damage inhibitor, the method including:
(a) culturing cells to obtain a cell suspension;
(b) gelating the cell suspension to prepare a cell-embedded gel;
(c) treating the cell-embedded gel with a DNA damaging agent and a DNA damage inhibitory candidate;
(d) lysing the cell-embedded gel;
(e) performing DNA glycosylase treatment on the product of step (d);
(f) labeling the product of step (e) through nick translation;
(g) extracting genomic DNA from the product of step (f); and
(h) analyzing the genomic DNA.

The “DNA damaging agent” in step (c) may be any material that induces DNA damage, known in the art, by any method known in the art. Examples of the DNA damaging agent include radiation, UV radiation, oxygen radicals, and hydrocarbons, but are not limited thereto.

The analysis in step (h) is determined as follows:

(i) If the DNA damage is less in the group treated with a DNA damaging agent and a DNA damage inhibitory candidate compared with the group treated with only the DNA damaging agent, the DNA damage inhibitory candidate is determined to have a DNA damage inhibitory effect.

(ii) If the DNA damage is similar or equal in the group treated with a DNA damaging agent and a DNA damage inhibitory candidate compared with the group treated with only the DNA damaging agent, the DNA damage inhibitory candidate is determined to have no DNA damage inhibitory effect.

Since the screening method uses the method for evaluating DNA damage by an analyte, descriptions of overlapping contents between the two methods will be omitted to avoid excessive complication of the specification.

Features and advantages of the present invention are summarized as follows:

(a) The present invention provides a method for evaluating DNA damage by an analyte and a method for screening a DNA damage inhibitor.

(b) The present invention can quantitatively evaluate the extent of DNA damage by an analyte through visualization.

### BRIEF DESCRIPTION OF THE DRAWINGS

The above and other objects, features, and advantages of the present invention will be more apparent from the following detailed description taken in conjunction with the accompanying drawings, in which:

FIGS. 1a and 1b illustrate the visualization of alcohol-induced DNA damage using Escherichia coli genomic DNA. FIG. 1a is a schematic diagram showing an experimental scheme for alcohol treatment of Escherichia coli. The Escherichia coli-embedded agarose gel inhibits shear-induced DNA damage. The next steps are denoted from gelation to visualization.

FIGS. 2a and 2b illustrate analysis results of ethanol-induced DNA damage in Escherichia coli. FIG. 2a shows single molecule DNA damage analysis using glycosylase mix (Fpg, Nfo, and Nei) represented by a circle. Each data point was obtained from 5-35 Mbps DNA image data. Error bars represent standard deviation from 3-7 experimental data sets. Linearity is 0.98 (r²). FIG. 2b illustrates serial dilution-spotting assays for E. coli with increasing ethanol concentration (0-40% and 15-19%). The second dataset clearly shows the reduced colonies with increasing ethanol.

FIGS. 3a and 3b illustrate alcoholic beverage-induced DNA damage in Escherichia coli. FIG. 3a shows single molecule DNA damage analysis results using glycosylase mix. The data are average of 3-7 experiments, and error bars represent standard deviation. FIG. 3b shows fluorescence microscopic images for damaged DNA molecules. Arrows indicate red spots representing damaged lesions labeled by AlexaFluor-647. Scale bars mean 20 μm (49 kbp).

FIG. 4a shows ESI-MS spectra (50-500 m/z) of clear rice wine (also known as Japanese sake) and citric acid using negative electrospray ionization scanning. The peak at 191 of citric acid represents [M-H]⁺peak, and 111 represents [M-H-CO₂-2H₂O]⁻. FIG. 4b illustrates single molecule DNA damage analysis results for the mix of 13% ethanol and citric acid (0.76 mM), compared with 13% ethanol, citric acid (0.76 mM), and clear rice wine.

### DETAILED DESCRIPTION

Hereinafter, the present invention will be described in detail with reference to examples. These examples are only for illustrating the present invention more specifically, and it will be apparent to those skilled in the art that the scope of the present invention is not limited by these examples.

### EXAMPLE

Materials and Methods

Chemicals

AlexaFluor-647 and YOYO-1 were purchased from Invitrogen ThermoFisher Scientific (Carlsbad, Calif.). Formamido pyrimidine DNA glycosylase (Fpg), endonuclease IV (Nfo), endonuclease VIII (Nei), proteinase K, yeast chromosome PFG marker and deoxyribonucleotide triphosphate (dNTPs) were purchased from New England Biolabs (Beverly, Mass.). DNA polymerase I was purchased from Roche Life Science (Indianapolis, Ind.). LB broth was purchased from Difco Laboratories (LB Broth, Miller). Ethanol (99.8%), EDTA and NaCl were purchased from Sigma-Aldrich (St Louis, Mo.). Low gelling temperature (LGT) agarose was purchased from Lonza (Rockland, Me.). N-trimethylsilylpropyl-N,N,N-trimethyl ammonium chloride and vinyl trimethoxy silane were purchased from Gelest (Tullytown, Pa.). Alcoholic beverages were Kloud (beer, 5%), Chungha (clear rice wine, 13%), Chamisul (soju, 20%), and Passport Scotch (whisky, 40%) purchased from a local convenience store.

Bacterial Growth

Escherichia coli K-12 MG1655 cells were grown in 5 mL LB broths in a shaking incubator (220 rpm) at 37° C. for six hours. Bacterial cells were harvested by centrifugation (10,000×g, 10 min) and washed twice with 0.85% NaCl solution and re-suspended in the same solution. The cell-suspension was then diluted such that OD600 was approximately between 0.5 and 1 and used for subsequent reactions.

Ethanol or Alcoholic Beverage Induced DNA Damage

Bacterial suspension (OD600=0.5) was mixed with 2% LGT agarose solution and then dispensed as 20 μL droplets on a surface and solidified in the refrigerator (4° C.) for 10 minutes. Then, bacteria embedded agarose plugs were incubated in ethanol or alcoholic beverages for 30 minutes at room temperature. For control experiments, ethanol (5%-40%) solutions were prepared by mixing 99.8% ethanol and 0.85% NaCl solution and used for incubating the bacteria embedded agarose plugs. After incubation, all these plugs were washed in 0.85% NaCl solution for half an hour.

Single Molecule Labeling

After alcohol treatment, bacteria embedded agarose plugs were subjected to lysis with proteinase K solution (50 units in 500 μL, Tris 10 mM and EDTA 1 mM, pH 8.0 (1×TE)) at 42° C. for 150 min. The plugs were then washed in 1 mL 1×TE overnight. For removing oxidized base adducts, agarose plugs were incubated with a mixture of 10 units Fpg, 10 units Nfo, and 20 units Nei in NEB buffer2 (50 mM NaCl, 10 mM Tris-HCl, 10 mM MgCl₂, 1 mM DTT, pH 7.9) at 37° C. for one hour. After enzyme treatment, the plugs were washed twice with 1 mL 1×TE buffer for half-an-hour. The plugs were then incubated with 5 units DNA Polymerase I, 1 mM AlexaFluor-647 labeled dUTP and dNTP mix (1 mM dATP, 1 mM dGTP, 100 μM dCTP, 100 μM dTTP) in the polymerase reaction buffer (50 mM Tris-HCl, 1 mM DTT, 10 mM MgCl₂, pH 7.5) at 37° C. for one hour to label gaps or nicks. In this step, we used specifically endonuclease free DNA polymerase I, because other DNA polymerases generated more labels under the same reaction conditions. After nick translation, the plugs were washed three times in 1 mL 1×TE buffer for one hour. Then agarose plugs were melted in 400 μL 1×TE buffer at 65° C. for 15 min and stained with 1 μM YOYO-1.

Glass Surface Preparation

Glass coverslips (22×22 mm) were racked in custom-made Teflon racks, cleaned by boiling in piranha solution (sulfuric acid and hydrogen peroxide 4:1) for 50 min, and rinsed extensively with deionized water until pH became neutral. Each coverslip was rinsed three times in ethanol (99.8%). Then, they were stored in ethanol in a polypropylene container at room temperature. For surface derivatization, 22 glass surfaces (22 mm×22 mm cover slips) were placed in a Teflon block holder in a clean container and allowed to dry for 10 min at room temperature. The derivatization solution was prepared by mixing 100 μL of N-trimethylsilylpropyl-N,N,N-trimethyl ammonium chloride into 250 mL water. The solution was poured into the container of 22 glass coverslips and incubated at 60° C. with 50 rpm of continuous shaking overnight. Finally, the surfaces were rinsed three times with water and ethanol and then stored in ethanol (99.8%).

PDMS Channel Preparation

A photoresist (SU-8 2005) template was created on the silicon wafer with each channel having dimensions of 100 μm (width)×5 μm (height)×1 cm (length). The mixture of PDMS and curing agent in a 10:1 ratio was poured onto the microchannel template on a silicon wafer and incubated for 3 hours at 65° C. After peeling, the PDMS microchannels were oxidized in air plasma conditions for 30 sec (CuteBasic, Femto, Korea). Then, PDMS was washed and stored in water.

DNA Mounting and Imaging

A PDMS device was mounted on the positively charged surface. Then, the solution of DNA molecules melted from low gelling temperature agarose plug was loaded onto the entrance of the microfluidic channels. While the solution moved through the microchannels by capillary action, DNA molecules were elongated and deposited on the positively charged surface. A solid-state 488 nm laser (Coherent Sapphire 488) was used to generate two colors of YOYO-1 and AlexaFluor-647 that were imaged with fluorescence resonance energy transfer (FRET), using 488-nm holographic notch filter for green channel and another emission filter (XF3076, Omega Optical, Brattleboro, Vt.) for the red channel. Image analysis was performed using ImageJ.

Serial Dilution Spotting Assay

The serial dilution-spotting assay was performed for alcohol susceptibility. 0.8 mL bacterial suspension was centrifuged at 10,000×g for 10 minutes and then cell pellet was re-suspended and incubated in ethanol or alcoholic beverages for 30 minutes. After incubation, the bacterial suspension was centrifuged at 10,000×g for 10 min. and the cell pellet was re-suspended in 990 μL LB media. LB media containing bacterial pellet was then serially diluted and spots for 10⁻², 10⁻³, 10⁻⁴, 10⁻⁵ and 10⁻⁶ dilutions were made by dispensing 5 μL of suspension on the LB plate. The cell culture plates were incubated at 37° C. overnight.

Mass Spectrometry

ESI-MS was performed on a Varian 500-MS LC ion-trap mass spectrometer (Palo Alto, Calif.). Mass spectra were acquired using an electrospray ionization source in the negative-ion mode. Rice wine and citric acid in methanol solution was directly injected into the mass spectrometer. Mass spectra were scanned from 50 to 500 m/z. Operating mass spectrometer parameters were like the followings: spray needle voltage, −5 kV; capillary voltage, −5000 V; drying temperature, 350° C.; drying nitrogen gas pressure, 30 psi; nebulizer air pressure, 35 psi; infusion flow rate, 200 μL/min.

Results and Discussion

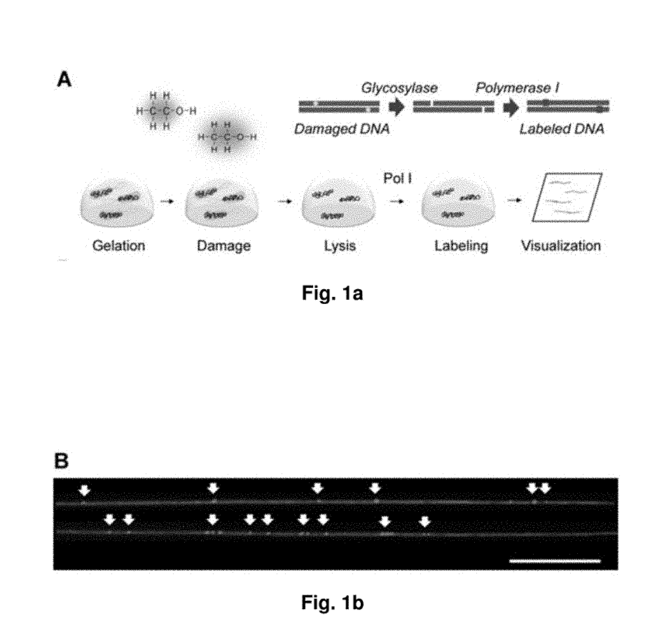

FIG. 1 illustrates the experimental scheme and representative fluorescent images for single-molecule visualization of alcohol-induced DNA damage. Bacterial cells were embedded in agarose gel, to prevent shear-induced mechanical stress to genomic DNA during cell lysis and subsequent biochemical reactions. Moreover, 100 nm pores in agarose gel allow enzymes and small molecules to freely pass through. DNA repairing enzyme, glycosylase recognize damaged part of DNA and generates single stranded breaks (SSB) in DNA by removing damaged nucleotides. These lesions were labelled with AlexaFluor-647-dUTP by nick translation using DNA polymerase I. Since DNA molecules were stained with the intercalating dye YOYO-1, the main detection principle is to use the fluorescence resonance energy transfer (FRET) between YOYO-1 and AlexaFluor-647 that shows DNA lesions as red dots shown in FIG. 1B. For visualization, DNA molecules were elongated and immobilized on the N-trimethylsilylpropyl-N,N,N-trimethyl ammonium coated positively charged surface within microfluidic channels after melting the low-gelling temperature agarose gel. Although the E. coli K-12 MG1655 strain genome was 4.6 Mbps long, procedures after gel melting generated DNA fragments. Therefore, E. coli genomic DNA fragments labelled and measured were visualized as 100-350 kb fragments. DNA size was determined from molecular length, calibrated with YOYO-1 stained T4 DNA length (68.6 μm for 166 kb).

First, we determined the number of intrinsic single strand breaks in E. coli genome. We found two labels out of 64 molecules that correspond to 15.9 Mbps from four different data sets. Therefore, we chose E. coli cells as a model system. Specifically, two data sets showed no labels and the other two data sets showed one label each. Based on these results, the control value for intrinsic SSB was set at 0.13 lesions/Mb or 0.58 lesions per E. coli genome (4.6 Mbps). This value is even smaller than our previous report in which two lesions from 122λ phage DNA molecules (48.5 kbp) purified from propagated phages, corresponding to 0.34 lesions/Mb. On the other hand, this control value was only valid for freshly growing log-phase bacteria. This value was found to be even more for fully grown or saturated stationary phase bacteria. To maintain optimal experimental conditions only log-phase bacterial cultures (OD600=0.5) were used for all the experiments in this study. In addition, we also attempted to use human cell line (HEK293), but found that the control, the number of DNA damaged lesions without ethanol, was too high to sensitively detect damaged lesions. Furthermore, it was not obvious whether human cells might have intrinsic damaged lesions or cell line as a kind of tumour might have more DNA damage than normal cells. Therefore, we chose E. coli as a biological model system.

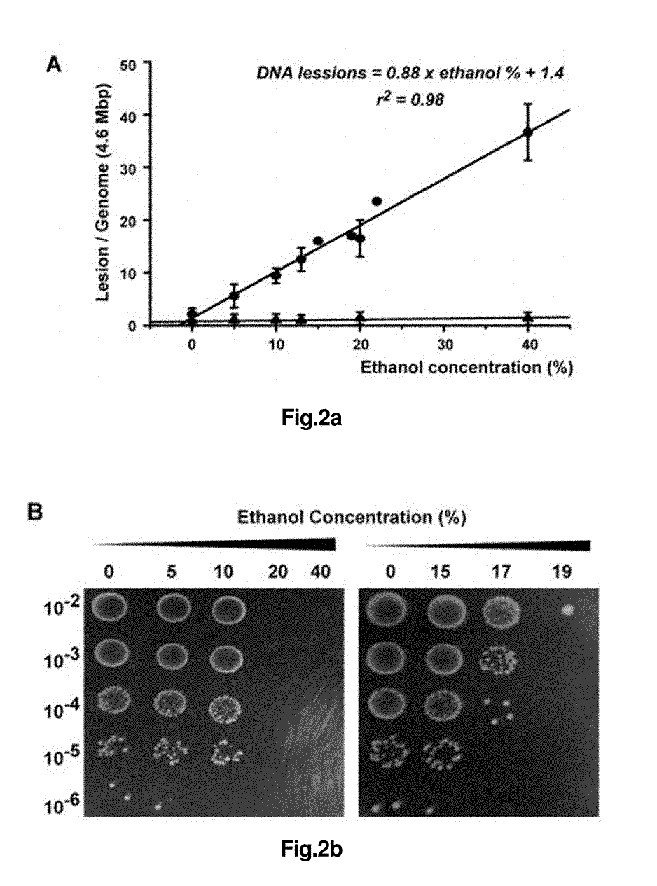

FIG. 2 demonstrates ethanol induced DNA damage on E. coli genome. It is quite intriguing that FIG. 2A shows a perfectly linear relationship (r²=0.98) between ethanol concentration and the number of DNA lesions, indicating that with every 1% increase in ethanol concentration, the number of lesions increased by 0.88 lesions/genome.

It is expected that one of critical toxicities of ethanol may originate from generating ROS such as superoxide anions and hydroxyl radicals, which cause oxidative damage to DNA. This assumption was the reason to utilize three glycosylases that recognize and remove oxidative DNA damage such as formamidopyrimidine-DNA-glycosylase (Fpg), endonuclease IV (Nfo), and endonuclease VIII (Nei). Our hypothesis was validated from the fact that ethanol incubation without glycosylase treatment generated only 1.1-1.4 labels per genome (triangles in FIG. 2A).

However, it is not clear how ROS-induced DNA damage occurs in E. coli. In eukaryotic cells, ethanol is oxidized to acetaldehyde by reducing nicotinamide dinucleotide (NAD⁺) to NADH, and then acetaldehyde is further oxidized to acetic acid with generation of another NADH by aldehyde dehydrogenase. Acetaldehyde itself can cause DNA damage directly, but ROS is more critical to DNA damage since the increased NADH concentration generates ROS via cellular respiratory system in the mitochondria.

In E. coli, ethanol is oxidized to acetaldehyde and further oxidized to acetyl-CoA with generation of two NADH molecules by aldehyde dehydrogenase, too. However, ethanol stress in E. coli makes the membrane more fluidic to cause membrane leakage. To the best of our knowledge, there has been no report for directly showing ROS generation by NADH accumulation in E. coli. Alternatively, Fe²⁺ bound aldehyde dehydrogenase is known to generate hydroxyl radicals, which suggests that the mechanism of ROS generation in E. coli may be different from eukaryotic cell.

Although we do not fully understand the mechanism of ROS generation, FIG. 2A demonstrates the fact that ethanol generates ROS, which cause DNA damage in E. coli. To further understand alcohol induced DNA damage, we performed serial dilution spotting assay (FIG. 2B), which showed that bacterial cells could not survive for 30 minutes in ethanol concentration above 17%, corresponding to 16.4 lesions/genome, i.e., one lesion per ˜300 kb. However, cell death was not only due to DNA damage, but also from combined effects of a variety of different physiological responses against ethanol stress such as the inhibition of peptidoglycan biosynthesis and fatty acid biosynthesis. In fact, ethanol acts via numerous mechanisms to affect the survival of bacterial cells. A recent study reported that there are considerable amount of proteome changes occurred by ethanol stress in E. coli. Nevertheless, FIG. 2 clearly demonstrates the strong correlation between DNA damage and bacterial cell death with the increase of ethanol concentrations (FIG. 2).

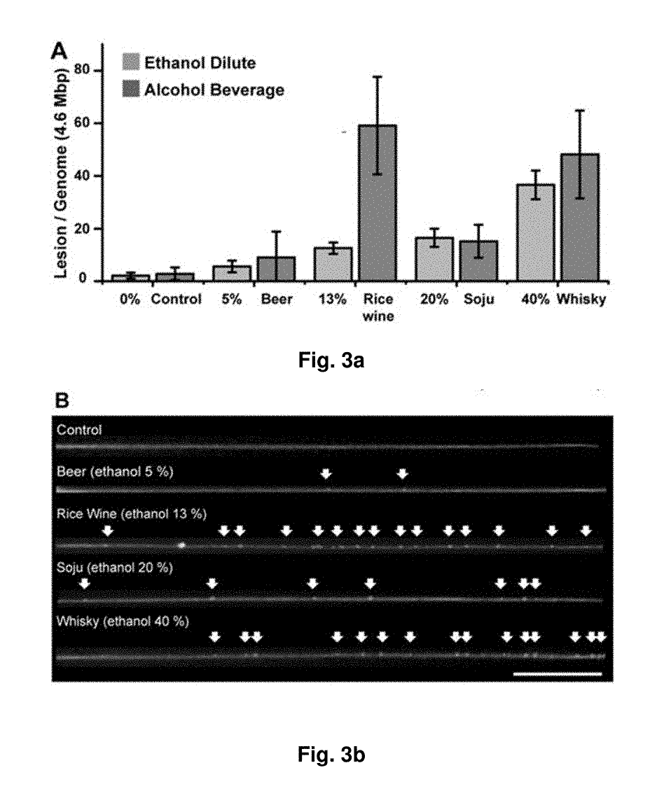

FIG. 3 demonstrates alcoholic beverage induced DNA damage by incubating E. coli embedded agarose plug in an alcoholic beverage for 30 minutes. Alcoholic beverages were used at ethanol concentrations of 5% beer, 13% clear rice wine (also known as sake), 20% soju (Korean liquor distilled from the wine fermented from various starch sources such as rice, wheat, potato, or tapioca, and further diluted with water), and 40% whiskey. For beer, soju, and whisky, the numbers of damaged lesions were similar to corresponding ethanol controls 5%, 20%, and 40%, while standard deviations were much larger than ethanol controls. However, the result for rice wine was conspicuous as the number of DNA lesions was considerably larger than with 13% ethanol.

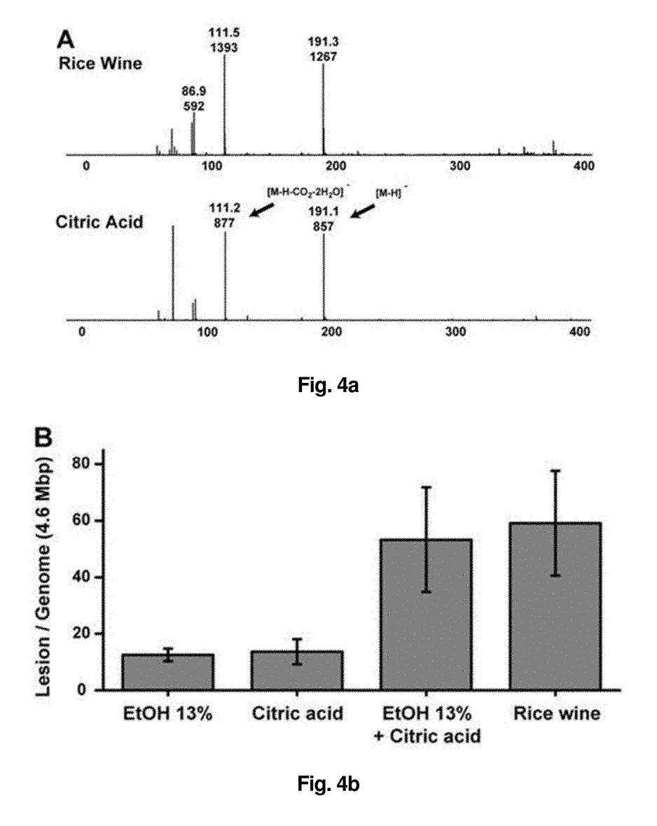

To obtain further insights and to analyze components of rice wine, we performed electrospray mass spectrometric analysis, which revealed that citric acid was the primary substance (FIG. 4A). In general, citric acid is a naturally occurring additive in most kinds of wine such as rice wine and grape wine. Importantly, it has antimicrobial activity since acid stress is well known to cause bacterial death. 30 Recent studies reported that citric acid could induce DNA damage in mammalian cells, too. Taken together, the DNA damage effect from rice wine represents how the components (mainly ethanol and citric acid), coherently damage DNA. To prove this effect, we performed DNA damage analysis using 13% ethanol titrated with citric acid (0.014%) to reach pH 3.24 to match that of rice wine. It is known that clear rice wine has 0.1% citric acid, but 0.1% of citric acid in 13% ethanol reduced pH to 2.77, probably due to other components. Since pH seems more critical factor, we used 13% ethanol solution matching the pH 3.24. Remarkably, this combination generated 53.3 lesions, which were quite close to 59.1 lesions by rice wine in the error range as shown in FIG. 4B.

Conclusions

In conclusion, we demonstrated the visualization of alcohol induced DNA damage using single molecule E. coli genomic DNA. This approach displayed extreme sensitivity that we were able to count the number of DNA damaged lesions, but also the ability to monitor physiological responses to toxic components. More importantly, the number of damaged lesions was linearly proportional to the increase of ethanol concentration. Using this approach, we evaluated alcoholic beverage induced DNA damage. Interestingly, we found enhanced DNA damage induced by citric acid, an additive of rice wine. Consequently, the visualization of DNA damage is powerful to quantitatively evaluate the extent of DNA damage by a toxic component, in a cell. Furthermore, integration of these reactions and visualization into a microfluidic system would promise the development of an effective biosensor.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method for evaluating DNA damage by an analyte, the method comprising:
(a) culturing cells to obtain a cell suspension;
(b) gelating the cell suspension to prepare a cell-embedded gel;
(c) bringing the cell-embedded gel into contact with an analyte;
(d) lysing the cell-embedded gel;
(e) performing DNA glycosylase treatment on the product of step (d);
(f) labeling the product of step (e) through nick translation;
(g) extracting genomic DNA from the product of step (f); and
(h) analyzing the genomic DNA.

2. The method of claim 1, wherein the cells in step (a) are selected from the group consisting of microorganisms, animal cells, and plant cells.

3. The method of claim 1, wherein the cell suspension in step (a) comprises log-phase bacteria.

4. The method of claim 1, wherein the gelating in step (b) is induced by adding agarose to a culture medium.

5. The method of claim 1, wherein the cell-embedded gel in step (b) has air holes of 10-1000 nm.

6. The method of claim 1, wherein the DNA glycosylase in step (e) is at least one selected from the group consisting of formamidopyrimidine [fapy]-DNA glycosylase (Fpg), endonuclease IV (Nfo), endonuclease VIII (Nei), 3-methyladenine DNA glycosylase II (AlkA), uracil-DNA glycosylase (UDG), endonuclease III (Nth), adenine DNA glycosylase (MutY), 3-methylpurine DNA glucosylase (AlkC), and akylpurine glycosylase D (AlkD).

7. The method of claim 1, wherein the labeling in step (f) is performed by DNA polymerase and a fluorescent-labeled dNTP mix (dATP, dCTP, dGTP, dTTP, and dUTP).

8. The method of claim 1, wherein the analyzing in step (h) is performed using a microfluidic device.

9. The method of claim 8, wherein the microfluidic device has a channel, into which a fluid is introduced, and a positively charged substrate directly connected with the channel.

10. A method for screening a DNA damage inhibitor, the method comprising:
(a) culturing cells to obtain a cell suspension;
(b) gelating the cell suspension to prepare a cell-embedded gel;
(c) treating the cell-embedded gel with a DNA damaging agent and a DNA damage inhibitory candidate;
(d) lysing the cell-embedded gel;
(e) performing DNA glycosylase treatment on the product of step (d);
(f) labeling the product of step (e) through nick translation;
(g) extracting genomic DNA from the product of step (f); and
(h) analyzing the genomic DNA.

11. The method of claim 10, wherein the cells in step (a) are selected from the group consisting of microorganisms, animal cells, and plant cells.

12. The method of claim 10, wherein the cell suspension in step (a) comprises log-phase bacteria.

13. The method of claim 10, wherein the gelating in step (b) is induced by adding agarose to a culture medium.

14. The method of claim 10, wherein the cell-embedded gel in step (b) has air holes of 10-1000 nm.

15. The method of claim 10, wherein the DNA glycosylase in step (e) is at least one selected from the group consisting of formamidopyrimidine [fapy]-DNA glycosylase (Fpg), endonuclease IV (Nfo), endonuclease VIII (Nei), 3-methyladenine DNA glycosylase II (AlkA), uracil-DNA glycosylase (UDG), endonuclease III (Nth), adenine DNA glycosylase (MutY), 3-methylpurine DNA glucosylase (AlkC), and akylpurine glycosylase D (AlkD).

16. The method of claim 10, wherein the labeling in step (f) is performed by DNA polymerase and a fluorescent-labeled dNTP mix (dATP, dCTP, dGTP, dTTP, and dUTP).

17. The method of claim 10, wherein the analyzing in step (h) is performed using a microfluidic device.

18. The method of claim 17, wherein the microfluidic device has a channel, into which a fluid is introduced, and a positively charged substrate directly connected with the channel.
